(19) United States
(12) Patent Application Publication
(10) Pub. No.: US 2017/0119574 A1
(43) Pub. Date: May 4, 2017

(54) PHASE CHANGE MATERIAL TEMPERATURE CONTROL DEVICE, SYSTEM AND METHOD

(71) Applicant: Imanano, Inc., Irvine, CA (US)

(72) Inventors: Brian Doherty, Irvine, CA (US); Faiz Chowdhury, Irvine, CA (US)

(73) Assignee: Imanano, Inc., Irvine, CA (US)

(21) Appl. No.: 15/344,203

(22) Filed: Nov. 4, 2016

Related U.S. Application Data
(60) Provisional application No. 62/250,922, filed on Nov. 4, 2015.

(51) Int. Cl. A61F 7/08

(52) U.S. Cl. CPC A61F 7/08

(57) ABSTRACT

In accordance with one embodiment of the present invention, the invention comprises a pliable enclosure containing a Phase Change Material (PCM) that maintains a nearly constant predetermined temperature while transmitting heat during phase change. The predetermined temperature is maintained which the PCM absorbs heat from the body of the patient, thus maintain the patient's body temperature at a nearly constant predetermined temperature for as long as the PCM material is transitioning phase. The pliable enclosure, which in an embodiment forms an enclosed volume, may be comprised of a shell which contains the PCM. The shell may be fabricated from polyethylene, polyurethane, or any other pliable material and may further comprise graphene covering on at least one of its surfaces to provide a tough exterior that is resistant to puncture by, for example, hypodermic needles, suture needles, scalpels, or other sharp instruments.

### CROSS REFERENCE TO RELATED APPLICATIONS AND INCORPORATION BY REFERENCE

This non-provisional patent application is a non-provisional of, and claims benefit of priority to, U.S. provisional patent application Ser. No. 62/250,922 filed in the United States Patent and Trademark Office (USPTO) on Nov. 4, 2015, titled PHASE CHANGE MATERIAL TEMPERATURE CONTROL DEVICE, SYSTEM AND METHOD, which is hereby incorporated herein by reference in its entirety.

### STATEMENT REGARDING FEDERALLY SPONSORED RESEARCH OR DEVELOPMENT

Not applicable.

### INCORPORATION-BY-REFERENCE OF MATERIAL SUBMITTED ON A COMPACT DISK

Not applicable.

### BACKGROUND OF THE INVENTION

1. Field of the Invention

The field of the invention relates generally to devices, systems and methods for maintaining a body of a person or object within a desired temperature range using phase change materials and methods. In an embodiment, the invention relates to devices, systems and methods for warming a body of a person. In an exemplary embodiment, the device, system and method of the invention may be utilized to maintain the body of a person at a constant desired and predetermined temperature while that person undergoes medical treatment, such as surgery, in which the immediate environment may be at a temperature that is uncomfortably cool for the patient, or while the patient is undergoing anesthesia, both of which tend to reduce the body temperature of the person. Thus the field of the invention is generally directed to thermoregulation of a body or other object using phase change materials and methods.

2. Background Art

The loss of body heat during surgery, and especially while a patient is undergoing anesthesia, can have negative consequences for a patient. Even mild hypothermia can be dangerous for patients. The consequences of even a few degrees Fahrenheit (Celsius) reduction in temperature include increased blood loss and transfusion requirement, surgical wound infection, and prolonged hospitalization and recovery. Studies have shown that virtually all surgical patients become hypothermic during surgery. Surgery patients are at risk for hypothermia due to exposure of large body surfaces for extended periods of time in a cool operating room. Patients who are anesthetized lose their ability to shiver, which is the body's natural way to attempt to correct hypothermia. Keeping patients warm reduces wound infection rates, decreases the likelihood of myocardial infarction, reduces mortality rates and can shorten the amount of time patients spend in the intensive care unit for post-operative care, and thus reduces their overall length of stay in a hospital or other health care facility.

One solution that has been used to combat hypothermia in surgical patients is the use of forced air warming gowns, which force warmed air around the body of a patient within a gown worn by the patient. Another solution that has been used to combat hypothermia in surgical patients is the use of forced air warming blankets, which circulates warm air in and through a pliable enclosure upon which the patient lies. A still further solution is the use of electric warming blankets.

However, these systems of the prior are difficult to regulate for temperature control, require electric power, are prone to failure due to the nature of the electromechanical systems used to force warmed air, spread infection by distributing airborne microorganisms around the body of a patient (in the case of the forced air warming gown), or are prone to puncture (in the case of the forced air warming blanket). Electric warming blankets require electric power, represent an electric shock hazard and complicate the surgical theater by requiring electric current supply cords and connections.

What is needed in the art, therefore, is a device, system and method for warming the body of a person, for example a person undergoing anesthesia during surgery.

### BRIEF SUMMARY OF THE INVENTION

The present invention comprises an apparatus and method that have one or more of the following features and/or steps, which alone or in any combination may comprise patentable subject matter.

The present invention overcomes the shortcomings of the prior art in that it eliminates the need for electromechanical components for controlling the temperature of a body, is reusable due to its ability to be sterilized, does not spread infection by distributing airborne microorganisms around the body of a patient, is easily temperature controlled, and does not require electric power.

In accordance with one embodiment of the present invention, the invention comprises a pliable enclosure containing a Phase Change Material (PCM) that maintains a nearly constant predetermined temperature while absorbing or yielding up heat during a phase transition. The predetermined phase transition temperature is maintained which the PCM absorbs heat from, or supplies heat to, the body of a patient or other object, thus maintaining the patient's body or object temperature at a nearly constant predetermined temperature for as long as the PCM material is transitioning phase.

The pliable enclosure, which in an embodiment forms an enclosed volume, may be comprised of a shell which contains the PCM. The shell may be fabricated from plastics such as polyethylene, polyurethane, or any other pliable material and may further comprise graphene covering on at least one of its surfaces to provide a tough exterior that is resistant to puncture by, for example, hypodermic needles, suture needles, scalpels, or other sharp instruments.

The phase change material temperature control device, system and method of the invention is impervious to most solvents, and is rechargeable and reusable.

In an embodiment of the phase change material temperature control pad intended for use in warming patients during surgery, the phase change material changes phase at a predetermined temperature between 90° Fahrenheit (32.222° Celsius) and 110° Fahrenheit (43.333° Celsius). In a still further preferred embodiment, the phase change material of the invention changes phase at 95° Fahrenheit (35° Celsius). The PCM may be selected such that any desired phase transition temperature may be achieved.

In an embodiment, the invention may further comprise a carrier that provides a platform for receiving a phase change material temperature control pad of the invention, and upon which a person may lie while being warmed by the phase change material temperature control pad of the invention. Such a carrier may be useful in numerous applications, one of which is during a surgical operation. In such a situation the body of a person is disposed upon a phase change material temperature control pad of the invention which is received by the carrier while the PCM is undergoing phase transition, thus creating a stable platform upon which a person may lie while their temperature is being controlled.

The phase change material warming pad in its broadest application has numerous applications and thus the claims and scope of the invention is not be construed as limited to only surgical applications.

### BRIEF DESCRIPTION OF THE DRAWINGS

The accompanying drawings, which are incorporated into and form a part of the specification, illustrate one or more embodiments of the present invention and, together with the description, serve to explain the principles of the invention. The drawings are only for the purpose of illustrating the preferred embodiments of the invention and are not to be construed as limiting the invention. In the drawings:

FIG. 1 depicts a cross section of a phase change material temperature control pad of an exemplary embodiment of the invention, depicting the phase change material captured within the pliable enclosure enclosed volume, and depicting an embodiment of the graphene covering overlying a portion of the pliable enclosure.

FIG. 2A depicts a perspective view of an embodiment of the carrier and phase change material temperature control pad, showing the placement of the change material temperature control pad into the carrier.

FIG. 2B depicts an end view of the carrier and phase change material temperature control pad of an exemplary embodiment of the invention, showing the placement of the change material temperature control pad into the carrier.

FIG. 3 depicts an exemplary embodiment of the invention placed on a surgical table for warming a patient, further showing a patient disposed upon the phase change material temperature control pad of an embodiment of the invention.

FIG. 4 depicts an exemplary embodiment of the carrier of the invention, in which the carrier comprises an expanded weave of plastic material comprised of plastic wire, which may be further defined as stiff plastic wire, and wherein the expanded weave of plastic wire acts as a compressible structure, covered by a covering such as vinyl.

FIG. 5 depicts a flow diagram of an exemplary method of the invention for controlling the temperature of the body of a patient undergoing medical treatment, which may include anesthesia, including the steps of determining a desired temperature to apply to the body of a person; selecting temperature control pad covering material and thickness, and determining the temperature control pad covering material and graphene coating thermal resistance; selecting PCM material for use in the temperature control pad for maintaining a constant pad temperature; providing a temperature control pad comprising the selected PCM enclosed within the selected temperature control pad covering material and thickness; charging the PCM material to a desired temperature; and causing a body of a person to be disposed upon the temperature control pad of the invention while the PCM is transitioning phase.

### DETAILED DESCRIPTION OF THE INVENTION

The following documentation provides a detailed description of the invention.

Although a detailed description as provided in the attachments contains many specifics for the purposes of illustration, anyone of ordinary skill in the art will appreciate that many variations and alterations to the following details are within the scope of the invention. Accordingly, the following preferred embodiments of the invention are set forth without any loss of generality to, and without imposing limitations upon, the claimed invention. Thus the scope of the invention should be determined by the appended claims and their legal equivalents, and not merely by the preferred examples or embodiments given.

As used herein, “phase change material” means a substance or mixture of substances with a heat of fusion which, when melting and solidifying at a phase transition, or phase change, temperature T enables the substance to absorb or release amounts of heat energy. Heat is absorbed or released when the substance changes state as from from solid to liquid, and vice versa, at the phase transition, or phase change, temperature T. T is a function of the physical properties of the phase change material. Phase change materials may comprise eutectic materials, salt hydrates, or organic materials. Eutectic materials tend to be solutions of salts in water that have a phase change temperature below 0° C. (32° F.). Salt hydrates are specific salts that are able to incorporate water of crystallization during their freezing process and tend to change phase above 0° C. (32° F.). Organic materials used as PCMs tend to be polymers with long chain molecules composed primarily of carbon and hydrogen. They tend to exhibit high orders of crystallinity when freezing and mostly change phase above 0° C. (32° F.). Examples of materials used as positive temperature organic PCMs include waxes, oils, fatty acids and polyglycols. The definition of “phase change material” or “PCM” further includes compositions comprised of more than one such substance, i.e., mixtures of phase change materials. Examples of substances which are phase change materials are shown in Table 1.

TABLE 1

1-Cyclohexylooctadecane
2-Heptadecanone
3-Heptadecanone
4-Heptadacanone
9-Heptadecanone
Acetamide
Acetic acid
Acrylic acid
Actanilide
Alpha napthol
Aluminum
Azobenzene
Bee wax
Bees wax
Benzamide
Benzoic acid
Benzylamine
Bromcamphor
Camphene
Camphenilone
Capric acid
Caprilic acid
Caprylone
Catechol
Cetyl acid
Chloroacetic acid
Copper
Cyanamide
Dinto toluent (2,4)
Diphenyl amine
Docasyl bromide
Durene
Eladic acid
Formic acid
Glautaric acid
Glycerin
Glycolic acid
Glyolic acid
Gold
Heptadecanone
Heptaudecanoic acid
Hydrocinnamic acid
Hypophosphoric acid
Iron
KNO3
KNO3 (10%)/NaNO3
KNO3/KBr (4.7%)/KCl (7.3%)
KNO3/KCl (4.5%)
KOH
Lauric acid
Lead
Lithium
Methly brombenzoate
Methyl behenate
Methyl eicosanate
Methyl fumarate
Methyl palmitate
Mn(NO₃)₂•6H₂O + MnCl₂•4H₂O (4% w/w)
Myristic acid^{[35]}
Na₂SiO₃•5H₂O
NaCl (26.8%)/NaOH
NaCl (42.5%)/KCl (20.5)/MgCl2
NaCl (5.0%)/NaNO3
NaCl (5.7%)/NaNO3 (85.5%)/Na2SO4
NaCl/NaNO3 (5.0%)
NaCl/KCL (32.4%)/LiCl (32.8%)
NaCl•Na₂SO₄•10H₂O
NaNO2
NaNO3
NaOH
NaOH/Na2CO3 (7.2%)
Nitro napthalene
O-Nitroaniline
O-Xylene dichloride
Oxolate
p-Bromophenol
p-Dichlorobenzene
p-Joluidine
p-Lattic acid
p-Xylene dichloride
Palmatic acid
Paraffin 14-Carbons
Paraffin 15-Carbons
Paraffin 16-Carbons
Paraffin 17-Carbons
Paraffin 18-Carbons
Paraffin 19-Carbons
Paraffin 20-Carbons
Paraffin 21-Carbons
Paraffin 22-Carbons
Paraffin 23-Carbons
Paraffin 24-Carbons
Paraffin 25-Carbons
Paraffin 26-Carbons
Paraffin 27-Carbons
Paraffin 28-Carbons
Paraffin 29-Carbons
Paraffin 30-Carbons
Paraffin 31-Carbons
Paraffin 32-Carbons
Paraffin 33-Carbons
Paraffin 34-Carbons
Pentadecanoic acid
Phenol
Phenylacetic acid
Polyethylene glycol 600
Quinone
Silver
Sodium sulfate (Na₂SO₄•10H₂O)
Stearic acid
Stibene
Succinic anhydride
Thiosinamine
Thymol
Titanium
TME (63% w/w) + H₂O (37% w/w)
Trimyristin
Tristearin
Water
Zinc
α-Chloroacetic acid
α-Nepthylamine
β-Chloroacetic acid

### Examplary Phase Change Materials

As used herein, “memory cell foam” means polyurethane with additional chemicals increasing its viscosity and density. It is often referred to as “viscoelastic” polyurethane foam, or low-resilience polyurethane foam (LRPu). Higher-density memory foam softens in reaction to body heat, allowing it to mold to a warm body in a few minutes. The invention may comprise any density memory foam, but may preferentially be comprised of memory foam that exhibits a density from less than 1.5 lb/ft³ to 8 lb/ft³ density.

As used herein, “plastic” means a material consisting of any of a wide range of synthetic or semi-synthetic organics that are malleable and can be molded into solid objects of diverse shapes. Plastics are typically organic polymers of high molecular mass, but they often contain other substances. They are usually synthetic, most commonly derived from petrochemicals, but many are partially natural. Examples of plastics include but are not limited to polyester (PES); polyethylene terephthalate (PET); polyethylene (PE); high-density polyethylene (HDPE); polyvinyl chloride (PVC); polyvinylidene chloride (PVDC) (Saran); low-density polyethylene (LDPE); polypropylene (PP); polystyrene (PS); high impact polystyrene (HIPS); polyamides (PA) (Nylons); acrylonitrile butadiene styrene (ABS); polyethylene/Acrylonitrile Butadiene Styrene (PE/ABS); polycarbonate (PC); polycarbonate/Acrylonitrile Butadiene Styrene (PC/ABS); polyurethanes (PU); maleimide/Bismaleimide; melamine formaldehyde (MF); plastarch material; phenolics (PF) or (phenol formaldehydes); polyepoxide (Epoxy); polyetheretherketone (PEEK); polyetherimide (PEI) (Ultem); polyimide; polylactic acid (PLA); polymethyl methacrylate (PMMA) (Acrylic); polytetrafluoroethylene (PTFE); urea-formaldehyde (UF); furan; silicone; and polysulfone, or any combination of these materials.

As used herein, “graphene” means an allotrope of carbon in the form of a two-dimensional, atomic-scale, hexagonal lattice in which one atom forms each vertex. A graphene covering or coating may comprise a plurality of single-atom layers. Graphene is the basic structural element of other allotropes of carbon, including graphite, charcoal, carbon nanotubes and fullerenes. It can also be considered as an indefinitely large aromatic molecule, the limiting case of the family of flat polycyclic aromatic hydrocarbons. Each graphene atom may have four bonds, one σ bond with each of its three neighbors and one π-bond that is oriented out of plane. The atoms are typically spaced about 1.42 Å apart. Graphene is often produced as a powder and as a dispersion in a polymer matrix. This dispersion is suitable for coatings and forming coverings. Graphene may be deposited using an ultrasonic spray, which may be an atomized spray, to create a homogeneous thin film layer of graphene.

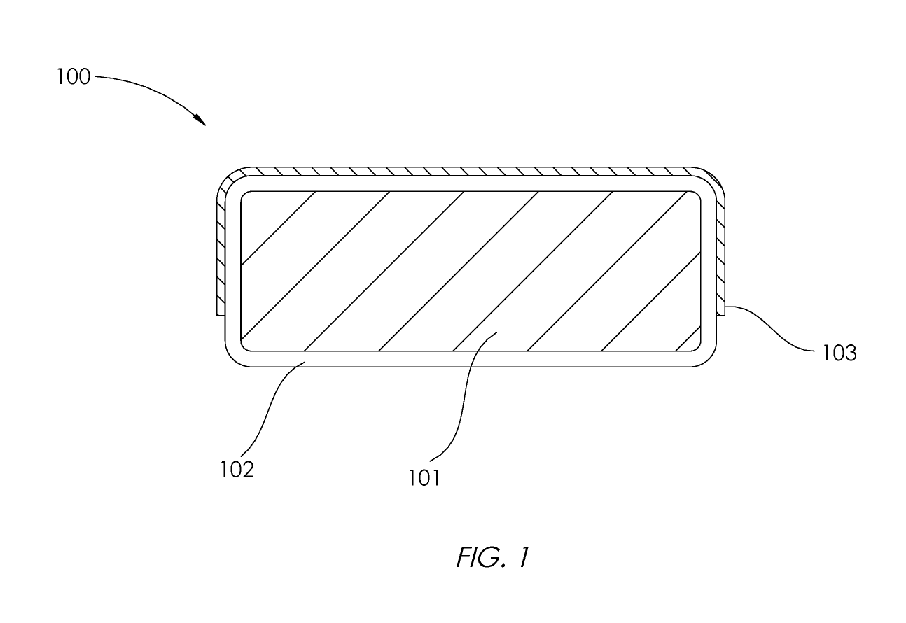

Referring now to FIG. 1, a cross section view of a phase change material temperature control pad 100 of an exemplary embodiment of the invention, depicting a phase change material 101 captured within a pliable enclosure forming an enclosed volume, and depicting an embodiment of a graphene covering overlying a portion of the pliable enclosure is depicted. Phase change material 101 is disposed in enclosure 102, which may be a pliable enclosure. Graphene coating 103 is attached to and covers a portion of enclosure 102. While a particular embodiment of graphene layer 103 is depicted in FIG. 1, graphene coating 103, which may be ultrasonically deposited on an exterior surface of pliable enclosure 102, may form a covering over any portion or all of enclosure 102. Pliable enclosure 102 material may be any pliable material that will contain a PCM material, but is typically a plastic material. Pliable enclosure 102 may be fabricated by any means known in the art. In the case in which pliable enclosure 102 is fabricated from plastic material, seams may be formed by fabricating the enclosure from separate pieces or sheets of pliable plastic in which the seams are closed and sealed by any means known in the art such as chemical bonding, heat sealing, ultrasonic welding, or the like.

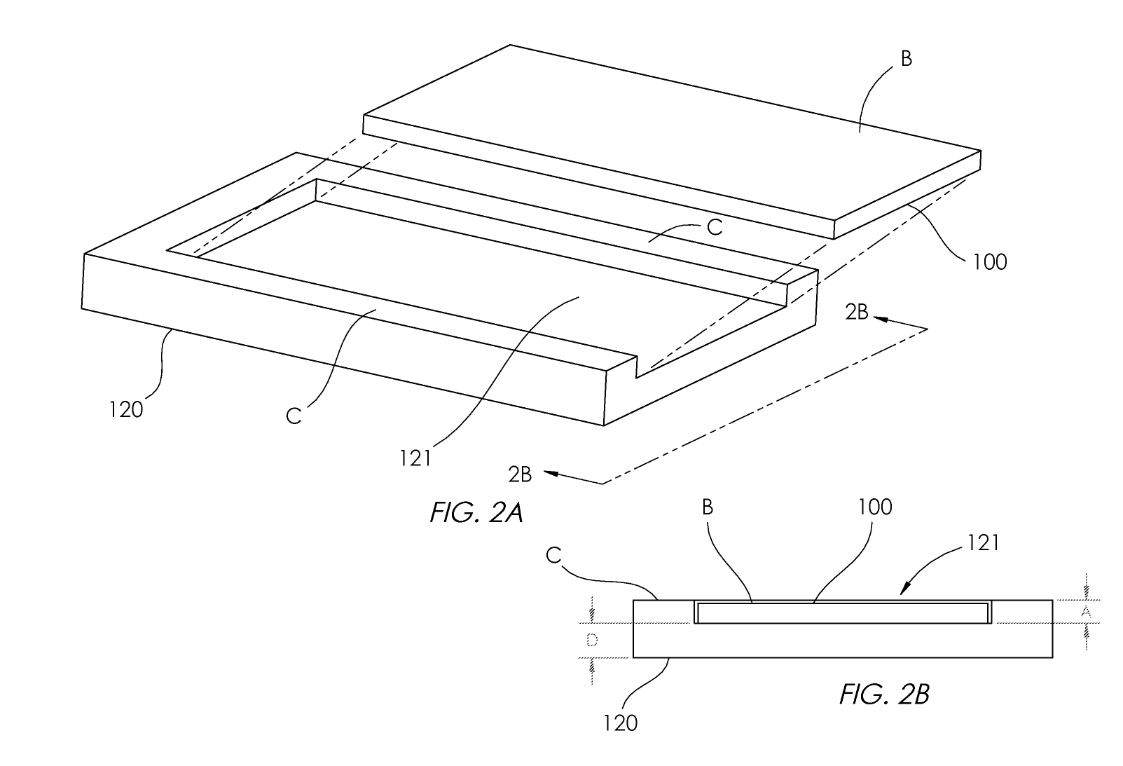

Referring now to FIG. 2A, a perspective view of the carrier and phase change material warming pad of an exemplary embodiment of the invention, showing the placement of the phase change material temperature control pad 100 into a carrier 120 of the invention is depicted. While a rectangular shape is depicted for both carrier 120 and the phase change material temperature control pad 100, both carrier 120 and phase change material temperature control pad 100 may take any shape desired, and in the preferred embodiments of the invention, recess 121 is shaped to a complementary shape matching the exterior shape of phase change material temperature control pad 100 such that recess 121 is adapted to receive phase change material temperature control pad 100 with a close fit. For example, and not by way of limitation, phase change material temperature control pad 100 may take on a hexagonal, square, circular or other shape, and recess 121 may take on a complementary matching shape such that phase change material temperature control pad 100 is received by recess 121 with a close fit.

Carrier 120 may be fabricated from any material, such as a compressible material, suitable for providing a compressible structure upon which a person may lie during surgery, such as, for example, open cell foam, closed cell foam or memory foam. In an alternative embodiment, carrier 120 may be fabricated from an expanded weave of plastic material comprised of plastic wire, which may be further defined as stiff plastic wire, and wherein the expanded weave of plastic wire acts as a compressible structure. Carrier 120 may be comprised of any compressible material, and may further comprise an outer covering fabricated from vinyl, fabric, rubberized fabric, or any other pliable mattress cover material known in the art. Preferably, the outer covering of carrier 120 comprises a waterproof, antimicrobial material. Upper surfaces C of carrier 120 may be, but are not necessarily, flush with upper surface B of phase change material temperature control pad 100 when phase change material warming pad 100 is placed within recess 121.

Referring now to FIG. 2B, an end view of the carrier and phase change material warming pad of an exemplary embodiment of the invention, showing the placement of the phase change material temperature control pad 100 into carrier 120 is depicted. Carrier 120 may comprise a recess 121 adapted to receive phase change material warming pad 100 as depicted. Recess 121 may be bordered on three, or fewer or more, sides by a raised surface C which may be disposed above the bottom surface of recess by a dimension A.

The phase change material temperature control pad 100 of the invention may have an upper surface B. Dimension A may be of such value that surface B is above, one the same plane as, or below surface C of carrier 120. In an exemplary embodiment, surface C may be coplanar with phase change material temperature control pad upper surface B.

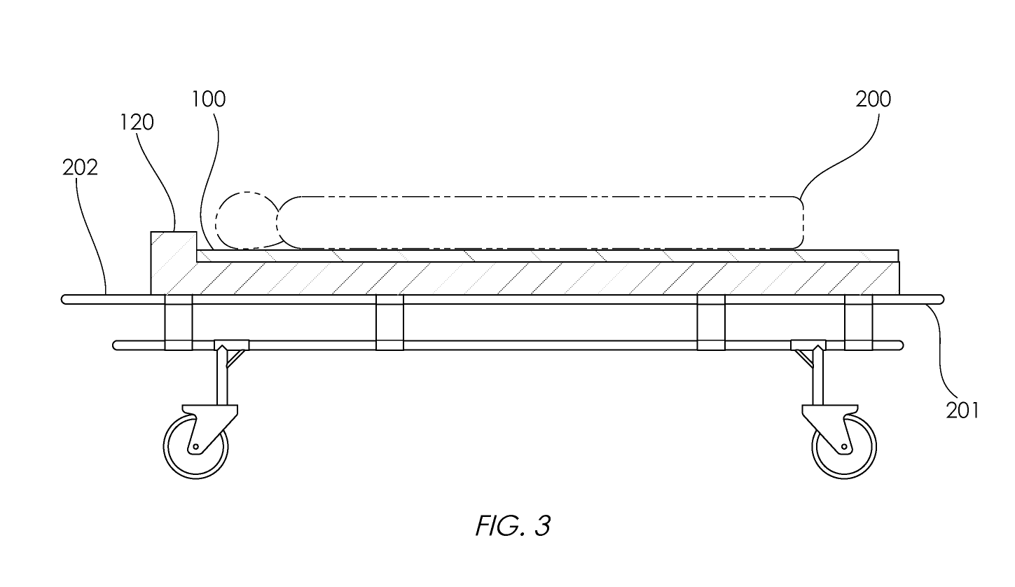

Referring now to FIG. 3, an exemplary embodiment of the invention placed on a surgical table for warming a patient, further showing a patient disposed upon the phase change material temperature control pad of an embodiment of the invention is depicted. A patient may be placed upon a phase change material temperature control pad 100 of the invention that has been received by recess 121 in a carrier of the invention 120. Typically, prior to use in a case where warming is desired, the temperature control pad 100 is “charged” to a temperature above a predetermined phase transition temperature. The predetermined phase transition temperature may be a temperature at which, given the temperature differential between the person's body and the PCM caused by the intervening material layers, which may comprise the temperature control pad 100 covering, graphene coating, and any other intervening materials, presents a desired temperature to the person's body while the PCM is transitioning states. For example, if it is desired to apply a temperature of 95° F. to a patient's body during the application of anesthesia during a medical procedure such as surgery, the thermal losses (and thus the temperature differential) between the patient's body and the PCM may be calculated using the known thermal properties of the intervening materials. In the example, assume a three degree (3°) F. drop in temperature. Thus it would be desired that the phase change material temperature control pad 100 of the invention transition phase at 98° F., and a suitable phase change material exhibiting this phase transition temperature T is selected. The temperature control pad 100 may then be charged to a temperature slightly above 98° F., causing the PCM material in phase change material temperature control pad 100 to liquefy. The phase change material temperature control pad 100 may then be placed into and received by recess 121 in carrier 120, and the patient may be placed upon phase change material temperature control pad 100. As the phase change material temperature control pad 100 cools to 98° F., it will begin to give off heat as the PCM transitions state from liquid to solid, and will continue to give off this heat for as long as the PCM continues to transition physical state. Thus, a constant temperature of 95° F., or any other temperature desired as determined by the selection of PCM or mixture of PCM comprising the invention, is applied to the patient's body for as long as the PCM continues to change state.

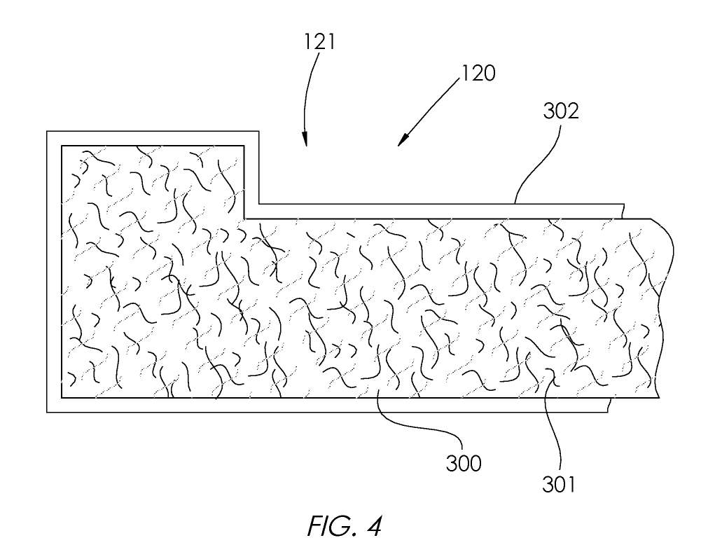

Referring now to FIG. 4, an exemplary embodiment of the carrier 120 of the invention is depicted in which the carrier comprises an expanded weave 300 of plastic material comprised of plastic wire 301, which may be further defined as stiff plastic wire, and wherein the expanded weave of plastic wire acts as a compressible structure. The expanded plastic weave which forms the carrier may be covered by a covering 302 such as vinyl or any other material which preferably, but not necessarily, does not absorb liquids.

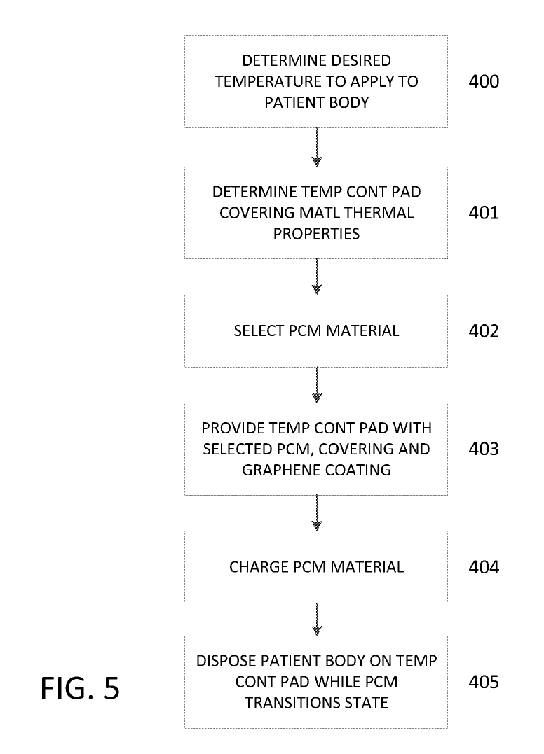

Referring now to FIG. 5, The present invention also comprises a method forming a series of steps for controlling the body temperature of a person, for example a person undergoing anesthesia during surgery, utilizing the phase change material temperature control pad 100 of the invention, comprising the following steps: 1) determining a desired temperature to apply to the body of a person in order to achieve a desired effect on the person 400; 2) selecting temperature control pad covering 102 material and thickness, and determining the temperature control pad covering material and graphene coating thermal resistance 401; 3) selecting PCM material for use in the temperature control pad for maintaining a constant pad temperature T while the PCM in the temperature control pad 100 is transitioning state, such that heat transferred to the body of a patient from the temperature control pad through the covering materials 102 and graphene coating, or heat transfer from the body of a patient to the temperature control pad through the covering materials 102 and graphene coating, results in a desired temperature being applied to the person's body given the thermal losses 402; 4) providing a temperature control pad comprising the selected PCM enclosed with the selected temperature control pad covering material and thickness 403; 5) charging the PCM material to a desired temperature that is above the PCM phase change temperature T in the case where it is desired to transfer heat to the body of a user from the temperature control pad, or charging the PCM material to a desired temperature that is below the PCM phase transition temperature T in the case where it is desired to transfer heat from the body of a user to the temperature control pad 404; and 6) causing the body of a person to be disposed upon the temperature control pad of the invention while the PCM material is undergoing phase transition 405.

In alternate embodiments of the method, more than one PCM may comprise the PCM utilized in the invention, resulting in the use of a PCM mixture, as may be necessary to achieve a specific desired PCM phase change temperature T, or to achieve any desired step function in, or duality of, PCM phase transition temperature T. In further alternate embodiments of the invention, one or more covering materials may be utilized, and each may be analyzed for thermal conductivity in order to achieve the desired thermal transfer between the body of a user and the temperature control pad.

What is claimed is:

1. A phase change material temperature control pad, comprising:
a pliable enclosure having an external surface and forming an enclosed volume;
a phase change material disposed within said enclosed volume of said pliable enclosure; and
a graphene coating covering at least a portion of said exterior surface of said pliable enclosure.

2. The phase change material temperature control of claim 1, wherein said phase change material exhibits phase transition at a predetermined temperature.

3. The phase change material temperature control pad of claim 2, wherein said predetermined temperature is between 90° Fahrenheit and 110° Fahrenheit.

4. The phase change material temperature control pad of claim 2, wherein said predetermined temperature is between 95° Fahrenheit.

5. The phase change material temperature control pad of claim 1, wherein said graphene coating is deposited on said exterior surface of said pliable enclosure using an ultrasonic atomizing spray.

6. The phase change material temperature control pad of claim 1, wherein said pliable enclosure is fabricated from polyethylene.

7. The phase change material temperature control pad of claim 1, wherein said pliable enclosure is fabricated from polyurethane.

8. A phase change material temperature control system, comprising:
a temperature control pad comprising:
a pliable enclosure having an external surface and forming an enclosed volume;
a phase change material disposed within said enclosed volume of said pliable enclosure; and
a graphene coating covering at least a portion of said exterior surface of said pliable enclosure;
wherein said temperature control pad has an external shape; and
a carrier comprising a recess for receiving said temperature control pad, said carrier comprising a compressible material.

9. The phase change material temperature control of claim 8, wherein said phase change material exhibits phase transition at a predetermined temperature.

10. The phase change material temperature control pad of claim 9, wherein said predetermined temperature is between 90° Fahrenheit and 110° Fahrenheit.

11. The phase change material temperature control pad of claim 9, wherein said predetermined temperature is 95° Fahrenheit.

12. The phase change material temperature control pad of claim 8, wherein said graphene coating is deposited on said exterior surface of said pliable enclosure using an ultrasonic atomizing spray.

13. The phase change material temperature control pad of claim 8, wherein said pliable enclosure is fabricated from polyethylene.

14. The phase change material temperature control pad of claim 8, wherein said pliable enclosure is fabricated from polyurethane.

15. The phase change material temperature control system of claim 8, wherein said compressible material is further defined as comprising memory foam.

16. The phase change material temperature control system of claim 8, wherein said compressible material is further defined as comprising closed cell foam.

17. The phase change material temperature control system of claim 8, wherein said compressible material is further defined as comprising an expanded weave of plastic material comprised of plastic wire forming a compressible structure.

18. A method for warming a surgical patient, comprising the steps of:
providing a phase change material temperature control pad comprising:
a pliable enclosure having an external surface and forming an enclosed volume;
a phase change material disposed within said enclosed volume of said pliable enclosure;
a graphene layer covering at least a portion of said exterior surface of said pliable enclosure; and
placing a patient upon said phase change material temperature control pad such that the patient is disposed on said phase change material temperature control pad while said phase change material is undergoing phase transition.

19. The method for warming a surgical patient of claim 18, wherein said step of providing a phase change material warming pad comprising is further defined as wherein said phase change material changes physical state at a predetermined temperature.

20. The method for warming a surgical patient of claim 18, wherein said step of providing a phase change material warming pad comprising is further defined as said predetermined temperature being between 90° Fahrenheit and 110° Fahrenheit.

21. The method for warming a surgical patient of claim 18, wherein said step of providing a phase change material warming pad comprising is further defined as said predetermined temperature being 95° Fahrenheit.

22. A method for controlling the body temperature of a person during a medical procedure, comprising:
determining a desired temperature to apply to the body of a person in order during a medical procedure;
determining a temperature control pad covering material thermal resistance using thermal properties of said temperature control pad materials;
selecting a PCM material for use in the temperature control pad for maintaining a constant thermal control pad temperature T while the PCM is transitioning physical state, such that heat transferred to the body of a patient from the temperature control pad through the temperature control pad materials, or heat transfer from the body of a patient to the temperature control pad through the temperature control pad materials, results in a desired temperature being applied to the person's body;
providing a temperature control pad comprising the selected PCM enclosed with the selected temperature control pad covering materials;
charging the temperature control pad comprising the selected PCM to a desired temperature that is above the PCM phase change temperature T in the case where it is desired to transfer heat to the body of a user from the temperature control pad, or charging the temperature control pad comprising the selected PCM to a desired temperature that is below the PCM phase change temperature T in the case where it is desired to transfer heat from the body of a user to the temperature control pad; and
causing a body of a person to be disposed upon the temperature control pad of the invention while the PCM is transitioning state.

23. The method of claim 22, wherein said phase transition temperature is further defined as between 90° Fahrenheit (32.222° Celsius) and 110° Fahrenheit (43.333° Celsius).

24. The method of claim 22, wherein said phase transition temperature is further defined as 95° Fahrenheit.

25. The method of claim 22, wherein said phase transition temperature is further defined as 98° Fahrenheit (36.666° Celsius).
